(19) United States
(12) Patent Application Publication
(10) Pub. No.: US 2026/0155213 A1
(43) Pub. Date: Jun. 4, 2026

(54) COMPUTER-READABLE RECORDING MEDIUM STORING QUANTUM CIRCUIT WEIGHT REDUCTION PROGRAM, INFORMATION PROCESSING DEVICE, AND QUANTUM CIRCUIT WEIGHT REDUCTION METHOD

(71) Applicant: Fujitsu Limited, Kawasaki-shi, Kanagawa (JP)

(72) Inventor: Satoshi IMAMURA, Kawasaki (JP)

(73) Assignee: Fujitsu Limited, Kawasaki-shi, Kanagawa (JP)

(21) Appl. No.: 18/974,853

(22) Filed: Dec. 10, 2024

(30) Foreign Application Priority Data
Mar. 5, 2024 (JP) 2024-033345

(51) Int. Cl. G16C 10/00; G06N 10/20; G06N 10/60; G16C 20/20

(52) U.S. Cl. CPC G16C 10/00; G06N 10/20; G06N 10/60; G16C 20/20

(57) ABSTRACT

A recording medium stores a program for causing a computer to execute processing including: calculating energy of a molecule by a VQE and acquiring a number of iterations of the VQE for a molecular structure of each interatomic distance in the molecule by using quantum circuit information that represents a second quantum circuit obtained by reducing a number of Rz gates in a first quantum circuit, in which, when a cumulative increase number of the number of iterations from a first number of a first interatomic distance to a second number a second interatomic distance longer than the first interatomic distance becomes a threshold or more, the quantum circuit information is changed to quantum circuit information that represents a third quantum circuit obtained by increasing the number of Rz gates of the second quantum circuit.

### CROSS-REFERENCE TO RELATED APPLICATION

This application is based upon and claims the benefit of priority of the prior Japanese Patent Application No. 2024-33345, filed on Mar. 5, 2024, the entire contents of which are incorporated herein by reference.

### FIELD

The embodiments discussed herein are related to a quantum circuit weight reduction program, an information processing device, and a quantum circuit weight reduction method.

### BACKGROUND

Quantum chemical calculation is a method of analyzing a structure and a property of a molecule from an electronic state, and calculation of energy of a molecule to be analyzed is basic processing. As one of quantum chemical calculation algorithms, there is a variational quantum eigensolver (VQE). The VQE is also one of candidates for a variational algorithm that may be executed in an intermediate-scale quantum device without error correction (sometimes referred to as noisy intermediate-scale quantum (NISQ)).

Japanese Laid-open Patent Publication No. 2021-081819 is disclosed as related art.

### SUMMARY

According to an aspect of the embodiments, a non-transitory computer-readable recording medium stores a quantum circuit weight reduction program for causing a computer to execute processing including: processing of performing, in ascending order of an interatomic distance, processing of calculating energy of a molecule by a variational quantum eigensolver (VQE) and acquiring a number of iterations of the VQE for a molecular structure of each of a plurality of interatomic distances among a plurality of atoms included in the molecule by using quantum circuit information that represents a second quantum circuit obtained by reducing a number of Rz gates for each angle parameter included in a first quantum circuit for weight reduction, in which, in a case where a cumulative increase number of the number of iterations from a first number of iterations of a first interatomic distance among the plurality of interatomic distances to a second number of iterations of a second interatomic distance longer than the first interatomic distance among the plurality of interatomic distances becomes a threshold or more, the quantum circuit information to be used is changed to quantum circuit information that represents a third quantum circuit obtained by increasing the number of Rz gates for each angle parameter of the second quantum circuit, and the energy is calculated by the VQE for a molecular structure of a third interatomic distance longer than the second interatomic distance among the plurality of interatomic distances.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a diagram illustrating an example of a first quantum circuit;

FIG. 2 is a diagram illustrating an example of a second quantum circuit;

FIG. 3 is a diagram illustrating an example of a relationship between rz_per_param and an error of a variational quantum eigensolver (VQE);

FIG. 4 is a diagram illustrating an example of a relationship between an interatomic distance and the error of the VQE;

FIG. 5 is a diagram for describing an information processing device of a first embodiment;

FIG. 6 is a diagram illustrating an example of a relationship between an interatomic distance and the number of iterations;

FIG. 7 is a block diagram illustrating a hardware example of an information processing device of a second embodiment;

FIG. 8 is a block diagram illustrating a functional example of the information processing device;

FIG. 9 is a flowchart illustrating an example of a procedure of processing of a quantum circuit weight reduction method;

FIG. 10 is a flowchart illustrating an example of a procedure of processing of calculating energy of a molecule by a VQE;

FIG. 11 is a diagram illustrating an example of weight reduction of a quantum circuit;

FIGS. 12A and 12B are diagrams illustrating relationships of an error of the VQE and the number of iterations with an interatomic distance of a hydrogen molecule; and

FIG. 13 is a diagram illustrating an example of a potential energy curve of the hydrogen molecule.

### DESCRIPTION OF EMBODIMENTS

In the VQE, a trial wave function representing an electronic state of a molecule is expressed using a quantum circuit (sometimes referred to as a variational quantum circuit) having a rotation angle of a rotation gate as an angle parameter. Such a quantum circuit or trial wave function may also be referred to as ansatz. Additionally, energy of the molecule is calculated using the quantum circuit. Processing of optimizing the angle parameter by classical processing and calculating the energy by the quantum circuit are repeated so as to minimize this energy. Accuracy and a calculation amount of the VQE greatly depend on a type of the ansatz. As an ansatz capable of highly accurate calculation, there is a unitary coupled cluster singles and doubles (UCCSD) ansatz.

Since before, in order to perform efficient material design, there has been proposed a technology of calculating a molecular feature amount that satisfies a desired material function and calculating a constituent material that implements the molecular feature amount.

Meanwhile, in the VQE, the energy may be calculated for a molecular structure of each of a plurality of interatomic distances between a plurality of atoms included in the molecule. At this time, as the interatomic distance becomes longer, potential accuracy of the quantum circuit may deteriorate, and the accuracy of the VQE may deteriorate.

In one aspect, an object of an embodiment is to suppress deterioration in accuracy of a VQE.

Hereinafter, modes for carrying out embodiments will be described with reference to the drawings.

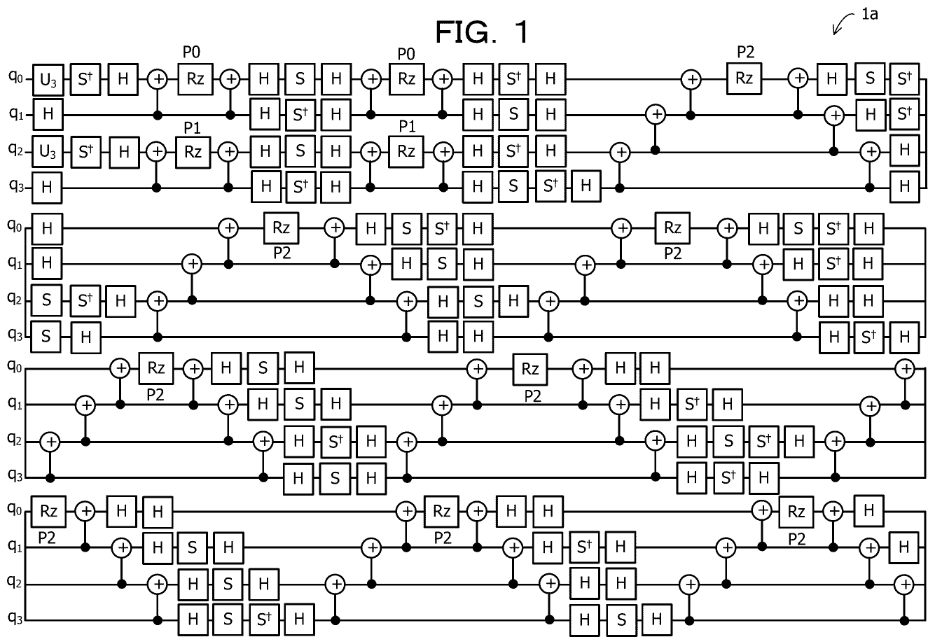

FIG. 1 is a diagram illustrating an example of a first quantum circuit. An example of a first quantum circuit 1a illustrated in FIG. 1 is a unitary coupled cluster singles and doubles (UCCSD) ansatz of a hydrogen (H₂) molecule represented by four qubits q₀ to q₃. Quantum operations on the qubits q₀ to q₃ by various quantum gates are indicated in four stages from the upper left to the lower right. A right end of each stage is coupled to a left end of the next stage.

In the first quantum circuit 1a, “U₃” is a U₃ gate that performs a rotation operation based on three angle parameters of θ, φ, and λ on a certain quantum state of a Bloch sphere. “S” is an S gate that performs a rotation operation of φ=π/2 on a certain quantum state of the Bloch sphere. “S” with a superscript dagger is an S-dagger gate that performs a rotation operation of φ=−π/2 on a certain quantum state of the Bloch sphere. “H” is an Hadamard gate that performs a rotation operation of 180° with an axis of inclination of 45° between a Z axis and an X axis as a rotation center on a certain quantum state of the Bloch sphere. “Rz” is an Rz gate that performs a rotation operation of θ about the Z axis on a certain quantum state of the Bloch sphere. “+” is a CNOT gate that inverts a value of a target qubit when a control qubit is |1>. The CNOT gate generates a quantum entanglement between two qubits.

In the first quantum circuit 1a, angle parameters of the Rz gate is represented by parameters P0, P1, and P2. In the example of FIG. 1, there are two Rz gates that perform the rotation operation using the parameter P0, there are also two Rz gates that perform the rotation operation using the parameter P1, and there are eight Rz gates that perform the rotation operation using the parameter P2.

The Rz gate is accompanied by a CNOT gate group. Each of the two Rz gates with the parameter P0 is accompanied by two CNOT gates, and each of the two Rz gates with the parameter P1 is also accompanied by two CNOT gates. Each of the eight Rz gates with the parameter P2 is accompanied by six CNOT gates.

In ideal simulation calculation without noise, the more the number of Rz gates for each angle parameter is, the more the angle parameter may be finely adjusted, so that an error of a variational quantum eigensolver (VQE) is small. However, in an actual quantum device with noise, in a case where the number of Rz gates is large, the error of the VQE tends to increase due to the noise. This is because, in a case where the number of Rz gates is large, the number of CNOT gates accompanying the Rz gates is also large. Since an error rate of the CNOT gate is particularly large, an increase width of the error due to the noise is larger as the number of CNOT gates in the quantum circuit is larger.

Therefore, by reducing the number of Rz gates, the number of CNOT gates accompanying the Rz gates may be reduced, and the error of the VQE due to the error caused by the noise may be reduced.

Hereinafter, the number of Rz gates for each angle parameter when the number of Rz gates is reduced for weight reduction from the first quantum circuit is referred to as rz_per_param. It is represented that the smaller a value of rz_per_param, the stronger a degree of the weight reduction of the quantum circuit.

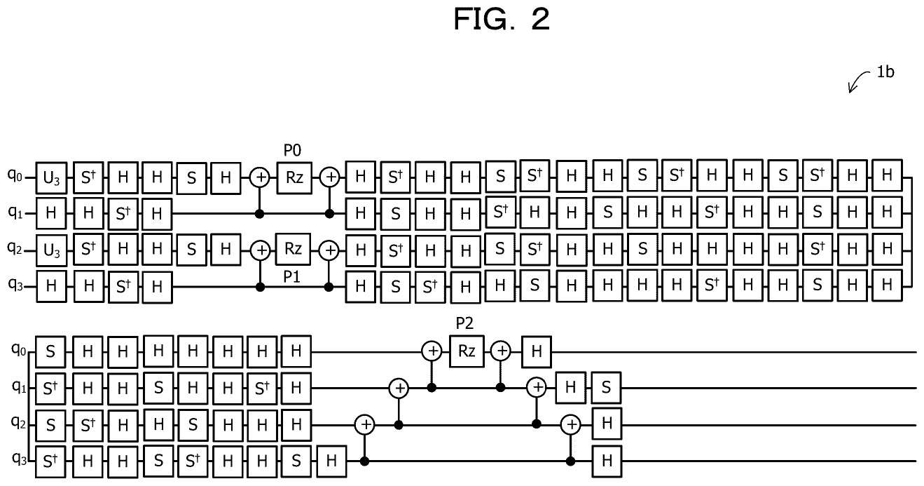

FIG. 2 is a diagram illustrating an example of a second quantum circuit. In the example of FIG. 2, a second quantum circuit 1b obtained by reducing the weight of the first quantum circuit 1a illustrated in FIG. 1 with the degree of weight reduction of rz_per_param=1 is indicated. From the second quantum circuit 1b, the plurality of CNOT gates accompanying the Rz gates deleted from the first quantum circuit 1a are also deleted.

As illustrated in FIG. 2, the second quantum circuit 1b has one Rz gate for each of the parameters P0 to P2. The number of CNOT gates is 56 in the first quantum circuit 1a, whereas it is 10 in the second quantum circuit 1b.

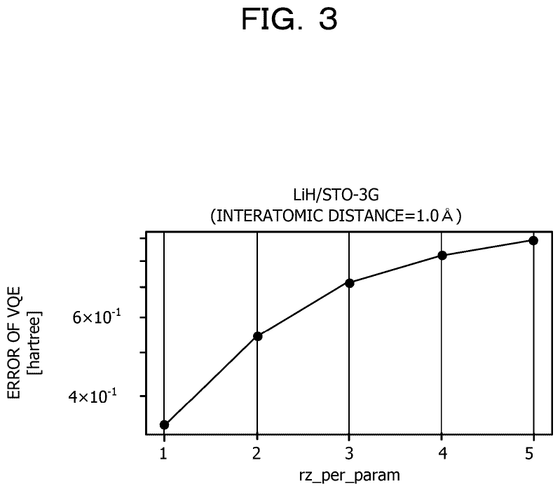

FIG. 3 is a diagram illustrating an example of a relationship between rz_per_param and the error of the VQE. A horizontal axis represents rz_per_param, and a vertical axis represents the error of the VQE.

Note that, in the example of FIG. 3, an ansatz of lithium hydride (LiH) is used. A basis set used is STO-3G. An interatomic distance between a lithium atom and a hydrogen atom used for calculation is 1.0 Å. Note that the error is a difference between base energy of LiH obtained by a full configuration interaction method and base energy of LiH obtained by VQE simulation with noise.

As illustrated in FIG. 3, the error of the VQE with noise may be reduced by decreasing the value of rz_per_param.

However, when energy is calculated for each interatomic distance of the molecule, as the interatomic distance becomes longer, potential accuracy of the quantum circuit may deteriorate and accuracy of the VQE may deteriorate.

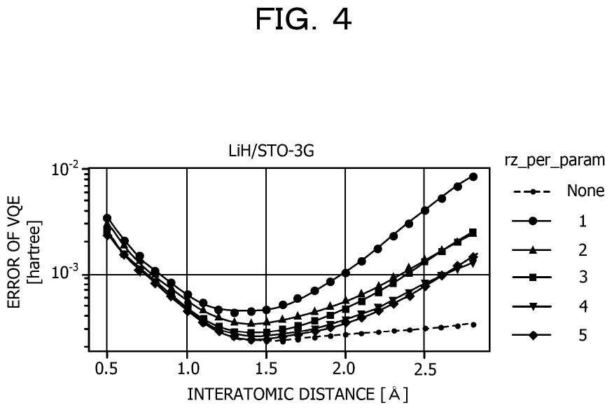

FIG. 4 is a diagram illustrating an example of a relationship between the interatomic distance and the error of the VQE. A horizontal axis represents the interatomic distance, and a vertical axis represents the error of the VQE.

Also in the example of FIG. 4, the ansatz of LiH and STO-3G are used. Note that the error is a difference between the base energy of LiH obtained by the full configuration interaction method and base energy of LiH obtained by VQE simulation without noise. The VQE simulation was performed for a case where there are five values of rz_per_param: 1, 2, 3, 4, and 5, and a case where weight reduction is not performed (referred to as “None”).

As illustrated in FIG. 4, when the interatomic distance is about 1.3 Å or more, the error of the VQE also increases as the interatomic distance becomes longer. This tendency becomes more remarkable as the value of rz_per_param decreases (for example, the degree of weight reduction increases).

Thus, in the embodiments indicated below, the value of rz_per_param is changed according to the interatomic distance at the time of calculation of the energy of the molecule by the VQE, thereby suppressing the deterioration of the accuracy of VQE.

### First Embodiment

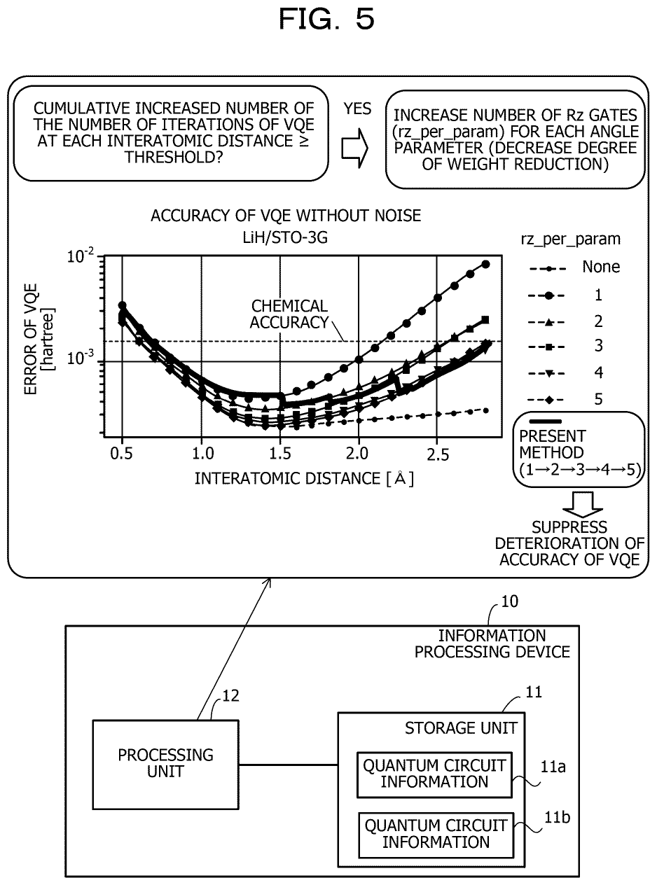

FIG. 5 is a diagram for describing an information processing device of a first embodiment.

An information processing device 10 of the first embodiment performs weight reduction of a quantum circuit and adjustment of a degree of the weight reduction (a value of rz_per_param) by processing to be described later, thereby suppressing deterioration of accuracy of a VQE. The information processing device 10 may be a client device or a server device. The information processing device 10 may be referred to as a computer.

The information processing device 10 includes a storage unit 11 and a processing unit 12. The storage unit 11 may be a volatile semiconductor memory such as a random access memory (RAM) or may be a nonvolatile storage such as a hard disk drive (HDD) or a flash memory. The processing unit 12 is, for example, a processor such as a central processing unit (CPU), a graphics processing unit (GPU), or a digital signal processor (DSP). Note that the processing unit 12 may include an electronic circuit such as an application specific integrated circuit (ASIC) or a field programmable gate array (FPGA). The processor executes, for example, a program (for example, a quantum circuit weight reduction program) stored in a memory such as a RAM (which may be the storage unit 11). A set of processors may be referred to as a multiprocessor or simply “processors”.

The storage unit 11 stores quantum circuit information 11a representing a second quantum circuit obtained by reducing the number of Rz gates for each angle parameter included in a first quantum circuit for weight reduction. In a case where a molecule is a hydrogen molecule, for example, the quantum circuit information 11a representing the second quantum circuit 1b as illustrated in FIG. 2 is stored.

The storage unit 11 may store the number of iterations of the VQE. In the VQE, calculation of energy and an energy gradient from a measurement result for the quantum circuit is repeated while updating the angle parameter until a predetermined convergence condition is satisfied. The number of times of repetition until the predetermined convergence condition is satisfied is the number of iterations.

As the convergence condition, for example, a condition may be used in which it is determined that convergence has occurred in a case where variation in a value of the angle parameter for obtaining base energy falls within a predetermined range. The number of iterations is related to the accuracy of the VQE. As the accuracy of the VQE deteriorates, the number of iterations until convergence increases.

The storage unit 11 may store energy of the molecule calculated by the VQE. Furthermore, the storage unit 11 may store the value of rz_per_param described above.

The processing unit 12 performs the following processing by executing the quantum circuit weight reduction program.

The processing unit 12 performs processing of calculating energy of a molecule by the VQE for a molecular structure of each of a plurality of interatomic distances between a plurality of atoms included in the molecule. In this processing, the processing unit 12 uses the second quantum circuit obtained by reducing the number of Rz gates for each angle parameter included in the first quantum circuit for weight reduction. The processing unit 12 performs the processing of calculating the energy and processing of acquiring the number of iterations of the VQE in ascending order of the interatomic distance.

The number of Rz gates for each angle parameter in the second quantum circuit, for example, an initial value of rz_per_param is preferably 1. This is because, as illustrated in FIG. 3, the smaller the value of rz_per_param (the higher the degree of weight reduction), the more the deterioration of the accuracy of the VQE due to noise may be suppressed.

In the first quantum circuit, in a case where there is a plurality of Rz gates with a certain angle parameter, for example, an Rz gate that acts earlier on a qubit is to be preferentially deleted. This is because an Rz gate that acts later is considered to be more important for a calculation result. Note that the processing unit 12 may specify the Rz gate to be preferentially deleted according to another rule.

The processing unit 12 acquires a cumulative increase number of the number of iterations from the first number of iterations of a first interatomic distance among the plurality of interatomic distances to the second number of iterations of a second interatomic distance longer than the first interatomic distance among the plurality of interatomic distances.

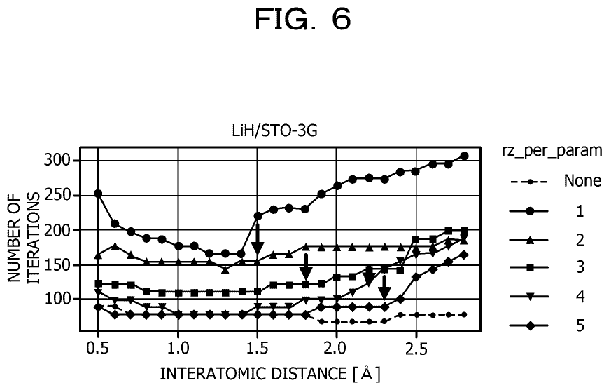

FIG. 6 is a diagram illustrating an example of a relationship between the interatomic distance and the number of iterations. A horizontal axis represents the interatomic distance, and a vertical axis represents the number of iterations.

Also in the example of FIG. 6, an ansatz of LiH and STO-3G are used. VQE simulation was performed for a case where there are five values of rz_per_param: 1, 2, 3, 4, and 5, and a case where weight reduction is not performed (referred to as “None”).

As illustrated in FIG. 4 described above, when the interatomic distance is about 1.3 Å or more, as the interatomic distance becomes longer, the number of iterations also increases as illustrated in FIG. 6, as well as an error of the VQE also increases.

In a case where the cumulative increase number is a threshold or more, the processing unit 12 changes the quantum circuit information 11a to be used to quantum circuit information 11b representing a third quantum circuit obtained by increasing the value of rz_per_param of the second quantum circuit. Using the quantum circuit information 11b, the processing unit 12 calculates the energy of the molecule by the VQE for a molecular structure of a third interatomic distance longer than the second interatomic distance.

For example, in the example of FIG. 6, it is assumed that the cumulative increase number of the number of iterations from the number of iterations when the interatomic distance is 1.4 Å to the number of iterations when the interatomic distance is 1.5 Å at rz_per_param=1 is the threshold or more. At this time, the processing unit 12 increases the value of rz_per_param of the second quantum circuit by, for example, 1, and calculates the energy of the molecule by the VQE for a molecular structure of the interatomic distance longer than 1.5 Å. In the example of FIG. 6, as the similar processing is repeated, as indicated by arrows, the value of rz_per_param is changed from 2 to 3, from 3 to 4, and from 4 to 5 as the interatomic distance becomes longer.

When increasing the value of rz_per_param, for example, the processing unit 12 sets the Rz gate that acts later on the qubit among the Rz gates deleted from the first quantum circuit to be preferentially added. This is because the Rz gate that acts later is considered to be more important for the calculation result. Note that the processing unit 12 may specify the Rz gate to be preferentially added according to another rule.

Note that, when a value of the threshold is too small, the value of rz_per_param is likely to increase, and conversely, when the threshold is too large, the value of rz_per_param is unlikely to increase. Therefore, it is preferable to set an appropriate value in consideration of accuracy to be satisfied (for example, chemical accuracy described later). Furthermore, an upper limit value of rz_per_param is smaller than the number of Rz gates for each angle parameter in the first quantum circuit.

In FIG. 5, the relationship between the interatomic distance and the error of the VQE illustrated in FIG. 4 is illustrated. In FIG. 5, as indicated by the arrows illustrated in FIG. 6, the relationship between the interatomic distance and the error of the VQE when the value of rz_per_param is changed is indicated by a thick line.

When rz_per_param=1 is kept, the error of the VQE increases as the interatomic distance becomes longer. On the other hand, by increasing rz_per_param as the interatomic distance becomes longer as in the method of the present embodiment (referred to as the present method in FIG. 5), an increase in the error of the VQE may be suppressed.

In quantum chemical calculation, it is preferable to keep the error within the chemical accuracy. Keeping the error within the chemical accuracy corresponds to decreasing the error smaller than 1.6 mHartree. As in FIG. 5, in a case where rz_per_param is fixed to 1, when the interatomic distance exceeds 2.1 Å, the error of the VQE exceeds the chemical accuracy. On the other hand, according to the method of the present embodiment, even when the interatomic distance is made longer to 2.8 Å, the error of the VQE may be kept within the chemical accuracy.

As described above, the information processing device 10 performs the processing of calculating the energy of the molecule by the VQE using the quantum circuit information 11a representing the second quantum circuit obtained by reducing the number of Rz gates for each angle parameter included in the first quantum circuit for weight reduction. The processing of calculating the energy of the molecule by the VQE is performed for the molecular structure of each of the plurality of interatomic distances between the plurality of atoms included in the molecule. The information processing device 10 performs the following processing when the processing of calculating the energy and the processing of acquiring the number of iterations of the VQE in ascending order of the interatomic distance. The information processing device 10 acquires the cumulative increase number of the number of iterations from the first number of iterations of the first interatomic distance among the plurality of interatomic distances to the second number of iterations of the second interatomic distance longer than the first interatomic distance among the plurality of interatomic distances. Then, in a case where the cumulative increase number is the threshold or more, the information processing device 10 changes the quantum circuit information 11a to be used to the quantum circuit information 11b representing the third quantum circuit obtained by increasing the number of Rz gates for each angle parameter of the second quantum circuit. Then, using the quantum circuit information 11b, the information processing device 10 calculates the energy of the molecule by the VQE for the molecular structure of the third interatomic distance longer than the second interatomic distance among the plurality of interatomic distances.

As a result, the increase in the error of the VQE due to the reduction in the number of Rz gates may be suppressed, and the accuracy of the VQE may be suppressed from deteriorating as the interatomic distance becomes longer. Furthermore, in a region where the interatomic distance is relatively short, since the cumulative increase number described above is less than the threshold, the number of Rz gates for each angle parameter does not increase, and the error of the VQE due to an error caused by noise may be reduced.

Note that, in the example described above, when the number of Rz gates for each angle parameter of the second quantum circuit is increased, the information processing device 10 calculates the energy by the VQE for the molecular structure of the third interatomic distance longer than the second interatomic distance. However, the information processing device 10 may calculate the energy by the VQE again using the quantum circuit information 11b representing the third quantum circuit obtained by increasing the number of Rz gates also for a molecular structure of the second interatomic distance.

### Second Embodiment

Next, a second embodiment will be described.

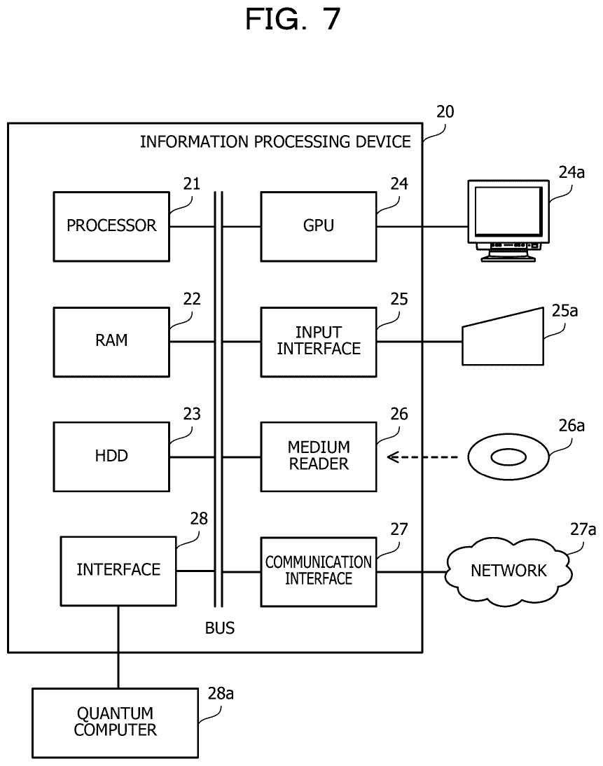

FIG. 7 is a block diagram illustrating a hardware example of an information processing device of the second embodiment.

An information processing device 20 may be a client device or a server device. The information processing device 20 may be referred to as a computer.

The information processing device 20 includes a processor 21, a RAM 22, an HDD 23, a GPU 24, an input interface 25, a medium reader 26, a communication interface 27, and an interface 28 that are coupled to a bus. The processor 21 corresponds to the processing unit 12 of the first embodiment. The RAM 22 or the HDD 23 corresponds to the storage unit 11 of the first embodiment.

The processor 21 executes a program command. The processor 21 loads a program such as a quantum circuit weight reduction program stored in the HDD 23 and data into the RAM 22, and executes the program. The processor 21 is a CPU, a GPU, a DSP, or the like. The information processing device 20 may include a plurality of processors.

The RAM 22 is a volatile semiconductor memory that temporarily stores a program to be executed by the processor 21 and data to be used by the processor 21 for arithmetic operations. The information processing device 20 may include a volatile memory of a type other than the RAM.

The HDD 23 is a nonvolatile storage that stores data and programs of software such as an operating system (OS), middleware, and application software. The information processing device 20 may include another type of nonvolatile storage such as a flash memory or a solid state drive (SSD).

The GPU 24 performs image processing in cooperation with the processor 21, and outputs an image to a display device 24a coupled to the information processing device 20. The display device 24a is, for example, a cathode ray tube (CRT) display, a liquid crystal display, an organic electroluminescence (EL) display, or a projector. Another type of output device such as a printer may be coupled to the information processing device 20.

Furthermore, the GPU 24 may be used as a general purpose computing on graphics processing unit (GPGPU). The GPU 24 may execute a program in response to an instruction from the processor 21. The information processing device 20 may include a volatile semiconductor memory other than the RAM 22 as a GPU memory.

The input interface 25 receives input signals from an input device 25a coupled to the information processing device 20. The input device 25a is, for example, a mouse, a touch panel, or a keyboard. A plurality of input devices may be coupled to the information processing device 20.

The medium reader 26 is a reading device that reads programs and data recorded in a recording medium 26a. The recording medium 26a is, for example, a magnetic disk, an optical disk, or a semiconductor memory. Examples of the magnetic disk include a flexible disk (FD) and an HDD. Examples of the optical disk include a compact disc (CD) and a digital versatile disc (DVD). The medium reader 26 copies the programs and data read from the recording medium 26a to another recording medium such as the RAM 22 or the HDD 23. The read program may be executed by the processor 21.

The recording medium 26a may be a portable recording medium. The recording medium 26a may be used for distribution of programs and data. Furthermore, the recording medium 26a and the HDD 23 may be referred to as computer-readable recording media.

The communication interface 27 communicates with another information processing device via a network 27a. The communication interface 27 may be a wired communication interface to be coupled to a wired communication device such as a switch or a router, or may be a wireless communication interface to be coupled to a wireless communication device such as a base station or an access point.

The interface 28 is coupled to a quantum computer 28a that executes processing of a quantum circuit, and reflects a value of an angle parameter determined by the processor 21 in the quantum circuit. Furthermore, the interface 28 acquires a result (measurement result) of the processing of the quantum circuit from the quantum computer 28a, and transmits the result to the processor 21.

As the quantum computer 28a, a quantum computer based on a quantum gate system may be used. As a qubit, for example, a qubit using a superconducting circuit, a qubit using an ion trap, a qubit using a light pulse, a qubit using a diamond color center, various types, and the like may be applied.

Next, functions of the information processing device 20 will be described.

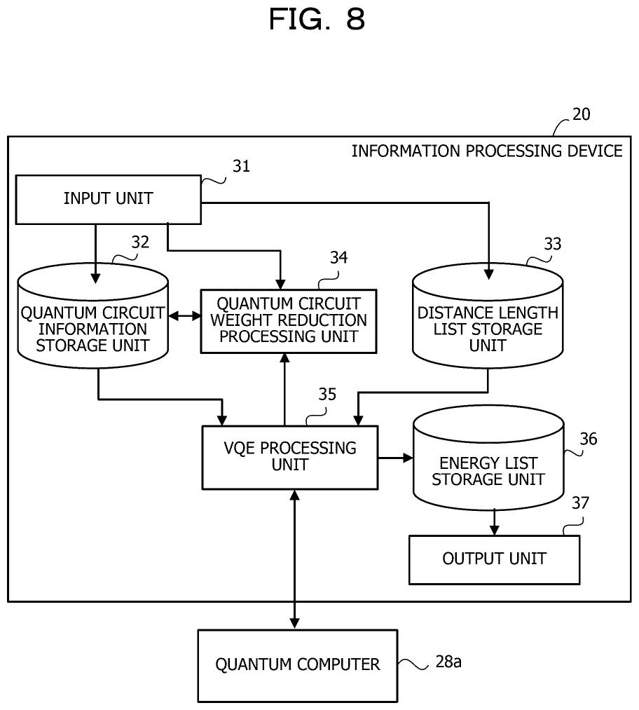

FIG. 8 is a block diagram illustrating a functional example of the information processing device.

The information processing device 20 includes an input unit 31, a quantum circuit information storage unit 32, a distance length list storage unit 33, a quantum circuit weight reduction processing unit 34, a VQE processing unit 35, an energy list storage unit 36, and an output unit 37.

Each storage unit described above is equipped using, for example, the RAM 22 or the HDD 23. The input unit 31, the quantum circuit weight reduction processing unit 34, the VQE processing unit 35, and the output unit 37 are equipped using, for example, the processor 21 and a program.

The input unit 31 receives input of information regarding a quantum circuit (quantum circuit information) representing an electronic state of a molecule whose energy is to be calculated by a VQE and input data such as a plurality of interatomic distances between a plurality of atoms included in the molecule. The quantum circuit includes a plurality of Rz gates and a plurality of CNOT gates accompanying each of the plurality of Rz gates. The input data may be input via, for example, the recording medium 26a or the network 27a, or may be input by an operation by a user using the input device 25a. Note that the information processing device 10 may generate the information regarding the quantum circuit.

The quantum circuit information storage unit 32 stores quantum circuit information.

The distance length list storage unit 33 stores a distance length list in which a plurality of input interatomic distances is arranged in ascending order of the interatomic distance.

The quantum circuit weight reduction processing unit 34 performs processing of reducing a weight of a quantum circuit by reducing the number of Rz gates and the number of CNOT gates accompanying the Rz gates included in the quantum circuit based on a value of rz_per_param. Moreover, the quantum circuit weight reduction processing unit 34 performs processing of increasing the value of rz_per_param in a case where a cumulative increase number of the number of iterations of the VQE performed in ascending order of the interatomic distance becomes a threshold or more.

The quantum circuit weight reduction processing unit 34 stores, for example, quantum circuit information representing the quantum circuit with a reduced weight or quantum circuit information representing the quantum circuit with the increased value of rz_per_param in the quantum circuit information storage unit 32 instead of the original quantum circuit information. Alternatively, the quantum circuit weight reduction processing unit 34 may store the quantum circuit information representing the quantum circuit with the reduced weight or the quantum circuit information representing the quantum circuit with the increased value of rz_per_param in the quantum circuit information storage unit 32 separately from the original quantum circuit information.

The VQE processing unit 35 performs processing of calculating energy of a molecule by the VQE. The processing of calculating the energy is performed, for example, as follows in ascending order of the interatomic distance based on a distance length list stored in the distance length list storage unit 33.

The VQE processing unit 35 reads quantum circuit information stored in the quantum circuit information storage unit 32, and causes the quantum computer 28a to execute processing of a quantum circuit with a reduced weight represented by the quantum circuit information. Then, the VQE processing unit 35 calculates the energy and an energy gradient of the molecule based on a processing result (measurement result), updates a value of an angle parameter of the quantum circuit until a predetermined convergence condition is satisfied, and causes the quantum computer 28a to repeat the processing of the quantum circuit. Then, the VQE processing unit 35 outputs the energy and the number of iterations when the predetermined convergence condition is satisfied.

The energy list storage unit 36 stores an energy list in which energy of a molecule having a molecular structure of each interatomic distance output by the VQE processing unit 35 is arranged.

The output unit 37 outputs the energy list. For example, the output unit 37 may output the energy list to the display device 24a and cause the display device 24a to display the energy list. The output unit 37 may transmit the energy list to another information processing device via the network 27a.

Next, a procedure of processing of a quantum circuit weight reduction method including the processing of calculating energy of a molecule by the VQE by the information processing device 20 will be described.

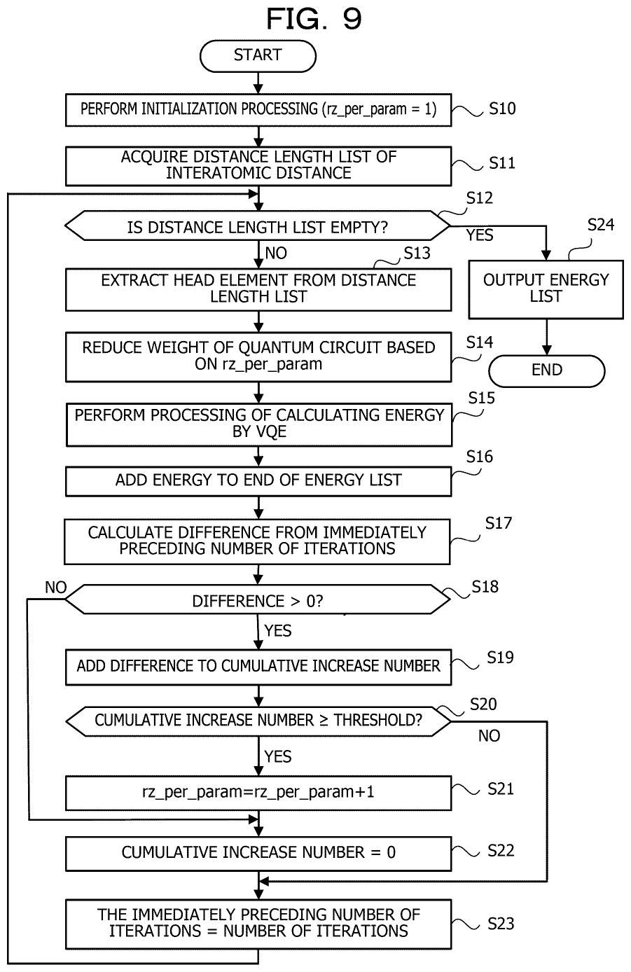

FIG. 9 is a flowchart illustrating an example of the procedure of the processing of the quantum circuit weight reduction method.

(Step S10) First, initialization processing is performed. In the initialization processing, the quantum circuit weight reduction processing unit 34 initializes rz_per_param to 1, the cumulative increase number of the iteration number to 0, and the immediately preceding number of iterations to 0. Furthermore, the energy list is initialized to empty.

(Step S11) The VQE processing unit 35 acquires the distance length list from the distance length list storage unit 33.

(Step S12) The VQE processing unit 35 determines whether or not the distance length list is empty. In a case where it is determined that the distance length list is not empty, processing of step S13 is performed, and in a case where it is determined that the distance length list is empty, processing of step S24 is performed.

(Step S13) The VQE processing unit 35 extracts a head element from the distance length list. Since the interatomic distances are arranged in ascending order of the interatomic distance in the distance length list, the head element of the distance length list is the shortest interatomic distance among the interatomic distances that have not yet been extracted.

(Step S14) The quantum circuit weight reduction processing unit 34 performs processing of reducing a weight of a quantum circuit based on a value of rz_per_param. Since an initial value of rz_per_param is 1, Rz gates and CNOT gates accompanying the Rz gates are deleted from the original quantum circuit so that the number of Rz gates for each angle parameter becomes one.

In a case where there is a plurality of Rz gates with a certain angle parameter, for example, the quantum circuit weight reduction processing unit 34 sets an Rz gate that acts earlier on a qubit to be preferentially deleted. This is because an Rz gate that acts later is considered to be more important for a calculation result. Note that the quantum circuit weight reduction processing unit 34 may specify the Rz gate to be preferentially deleted according to another rule.

In a case where rz_per_param is increased by processing to be described later, for example, the quantum circuit weight reduction processing unit 34 sets the Rz gate that acts later on the qubit among the Rz gates deleted from the original quantum circuit to be preferentially added. Note that the quantum circuit weight reduction processing unit 34 may specify the Rz gate to be preferentially added according to another rule.

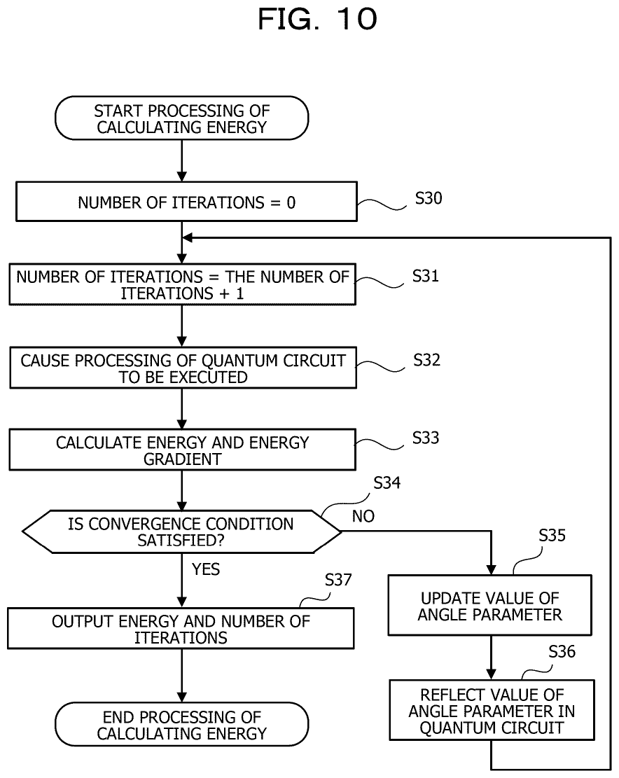

(Step S15) The VQE processing unit 35 performs processing of calculating energy of a molecule by the VQE on a molecular structure of the interatomic distance extracted in the processing of step S13 by using quantum circuit information representing the quantum circuit with a reduced weight. A procedure of the processing of calculating the energy will be described later (see FIG. 10).

(Step S16) The VQE processing unit 35 adds the energy calculated in the processing of step S15 to an end of the energy list stored in the energy list storage unit 36.

(Step S17) The quantum circuit weight reduction processing unit 34 calculates a difference between the immediately preceding number of iterations of the VQE and the current number of iterations of the VQE (=the current number of iterations of the VQE−the immediately preceding number of iterations of the VQE).

(Step S18) The quantum circuit weight reduction processing unit 34 determines whether or not the difference is larger than 0. In a case where it is determined that the difference is larger than 0, processing of step S19 is performed, and in a case where it is determined that the difference is 0 or less, processing of step S22 is performed.

(Step S19) The quantum circuit weight reduction processing unit 34 adds the difference calculated in the processing of step S17 to the cumulative increase number.

(Step S20) The quantum circuit weight reduction processing unit 34 determines whether or not the cumulative increase number is a predetermined threshold or more. In a case where it is determined that the cumulative increase number is the threshold or more, processing of step S21 is performed, and in a case where it is determined that the cumulative increase number is less than the threshold, processing of step S23 is performed.

(Step S21) The quantum circuit weight reduction processing unit 34 increases rz_per_param by 1.

(Step S22) The quantum circuit weight reduction processing unit 34 returns the cumulative increase number to 0.

(Step S23) The quantum circuit weight reduction processing unit 34 sets the immediately preceding number of iterations as the current number of iterations of the VQE. Thereafter, the processing of step S12 is performed again.

(Step S24) The output unit 37 outputs the energy list. As a result, the processing of the quantum circuit weight reduction method including the processing of calculating the energy of the molecule by the VQE ends.

FIG. 10 is a flowchart illustrating an example of the procedure of the processing of calculating the energy of the molecule by the VQE.

(Step S30) The VQE processing unit 35 initializes the number of iterations to 0.

(Step S31) The VQE processing unit 35 sets the number of iterations to the number of iterations+1.

(Step S32) The VQE processing unit 35 causes the quantum computer 28a to execute processing of the quantum circuit with the reduced weight.

(Step S33) The VQE processing unit 35 calculates the energy and an energy gradient of the molecule based on a processing result (measurement result) of the quantum circuit.

(Step S34) The VQE processing unit 35 determines whether or not a predetermined convergence condition is satisfied. In a case where it is determined that the convergence condition is not satisfied, processing in step S35 is performed, and in a case where it is determined that the convergence condition is satisfied, processing in step S37 is performed.

(Step S35) The VQE processing unit 35 updates a value of the angle parameter by a gradient descent method using the energy gradient calculated in the processing of step S33. In the gradient descent method, the value of the angle parameter is updated in a direction in which a decrease in the energy becomes maximum.

(Step S36) The VQE processing unit 35 reflects the updated value of the angle parameter in the quantum circuit. Thereafter, the processing of step S31 is performed again.

(Step S37) The VQE processing unit 35 outputs the energy and the number of iterations when the convergence condition is satisfied. As a result, the processing of calculating the energy of the molecule for the molecular structure of a certain interatomic distance ends.

Note that the order of the processing illustrated in FIGS. 9 and 10 is an example, and the order may be appropriately changed. Furthermore, the method of optimizing the value of the angle parameter is not limited to the gradient descent method as described above. The convergence condition may vary depending on the method of optimization to be applied.

Furthermore, in the example described above, in a case where the cumulative increase number is the threshold or more, the quantum circuit weight reduction processing unit 34 increases rz_per_param by one, but the number is not limited to one, and may increase rz_per_param by two or more.

As described above, the VQE processing unit 35 performs the processing of calculating the energy of the molecule by the VQE using the second quantum circuit (quantum circuit with a reduced weight) obtained by reducing the number of Rz gates for each angle parameter included in the first quantum circuit (original quantum circuit) for weight reduction. The processing of calculating the energy of the molecule by the VQE is performed for the molecular structure of each of the plurality of interatomic distances between the plurality of atoms included in the molecule, which is represented by the distance length list. Furthermore, the processing of calculating the energy by the VQE processing unit 35 and the processing of acquiring the number of iterations of the VQE by the quantum circuit weight reduction processing unit 34 are performed in ascending order of the interatomic distance. Moreover, the quantum circuit weight reduction processing unit 34 acquires the cumulative increase number of the number of iterations from the first number of iterations of the first interatomic distance among the plurality of interatomic distances to the second number of iterations of the second interatomic distance longer than the first interatomic distance among the plurality of interatomic distances. Then, in a case where the cumulative increase number is the threshold or more, the quantum circuit weight reduction processing unit 34 changes the quantum circuit information to the quantum circuit information representing the third quantum circuit obtained by increasing the number of Rz gates (value of rz_per_param) for each angle parameter of the second quantum circuit. The VQE processing unit 35 calculates the energy by the VQE for the molecular structure of the third interatomic distance longer than the second interatomic distance by using the quantum circuit information representing the third quantum circuit.

As a result, similarly to the first embodiment, an increase in an error of the VQE due to the reduction in the number of Rz gates may be suppressed, and accuracy of the VQE may be suppressed from deteriorating as the interatomic distance becomes longer. Furthermore, in a region where the interatomic distance is relatively short, since the cumulative increase number described above is less than the threshold, the number of Rz gates for each angle parameter does not increase, and the error of the VQE due to an error caused by noise may be reduced.

### (Example of Weight Reduction of Quantum Circuit)

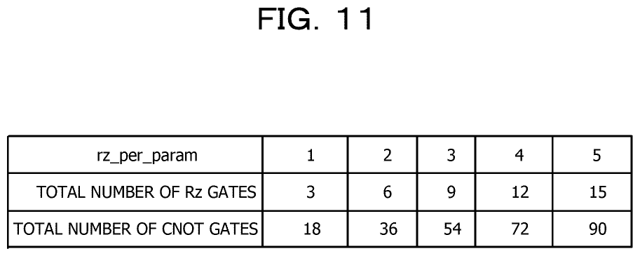

FIG. 11 is a diagram illustrating an example of weight reduction of a quantum circuit. In FIG. 11, an example of the weight reduction in a case where the number of angle parameters of an original quantum circuit is three, the number of Rz gates per angle parameter is eight, and the number of CNOT gates per Rz gate is six is illustrated. Therefore, in the original quantum circuit, the total number of Rz gates is 24, and the total number of CNOT gates is 144.

The smaller the value of rz_per_param, the higher the strength of the weight reduction. For example, in a case where rz_per_param=1 holds, the total number of Rz gates is three, and the total number of CNOT gates is 18. By reducing the number of CNOT gates having a large error rate, the error of the VQE due to an error caused by noise may be reduced.

(Example in which Cumulative Increase Number is not Threshold or More)

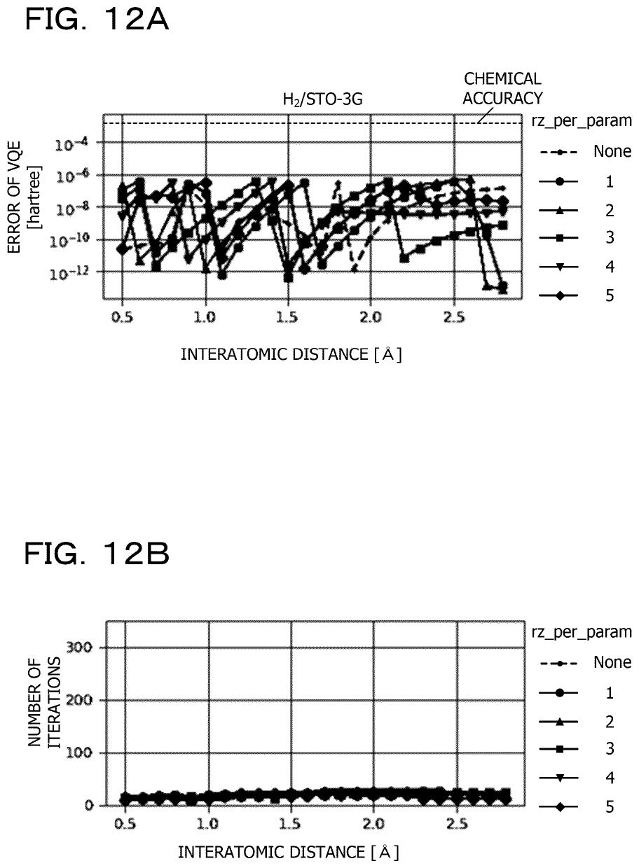

FIGS. 12A and 12B are diagrams illustrating relationships of an error of the VQE and the number of iterations with an interatomic distance of a hydrogen molecule. FIG. 12A is a diagram illustrating an example of the relationship between the interatomic distance of the hydrogen molecule and the error of the VQE, and FIG. 12B is a diagram illustrating an example of the relationship between the interatomic distance of the hydrogen molecule and the number of iterations. In FIG. 12A, a horizontal axis represents the interatomic distance, and a vertical axis represents the error of the VQE. In FIG. 12B, a horizontal axis represents the interatomic distance, and a vertical axis represents the number of iterations.

In the examples of FIGS. 12A and 12B, an ansatz of the hydrogen molecule and STO-3G are used. Note that the error is a difference between base energy of the hydrogen molecule obtained by the full configuration interaction method and base energy of the hydrogen molecule obtained by VQE simulation without noise. The VQE simulation was performed for a case where there are five values of rz_per_param: 1, 2, 3, 4, and 5, and a case where weight reduction is not performed (referred to as “None”).

In the case of the hydrogen molecule, as in FIG. 12A, there is no tendency that the error of the VQE increases as the interatomic distance becomes longer, and the error falls within chemical accuracy. As in FIG. 12B, the number of iterations also hardly changes even when the interatomic distance becomes longer. In such a case, the cumulative increase number of the number of iterations is not the threshold or more, and rz_per_param remains at the initial value of 1 even when the interatomic distance becomes longer. Therefore, a quantum circuit having the highest degree of weight reduction and being unlikely to be affected by noise may be used.

Note that, in the case of most molecules, it is considered to indicate a property that the error of the VQE increases as rz_per_param decreases as the interatomic distance becomes longer, as in LiH described above.

### (Output Example)

The output unit 37 may cause the display device 24a to display the following potential energy curve.

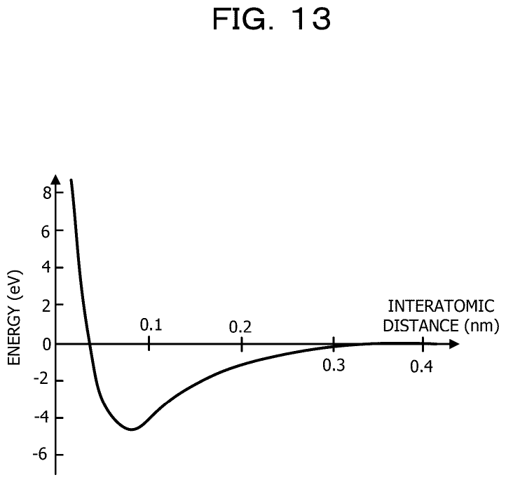

FIG. 13 is a diagram illustrating an example of a potential energy curve of the hydrogen molecule. In FIG. 13, a horizontal axis represents the interatomic distance, and a vertical axis represents energy (base energy).

The potential energy curve as in FIG. 13 is obtained by coupling energy plots calculated by the VQE for each interatomic distance with a line. A minimum point represents a stable state, and a maximum point represents a transition state.

By obtaining such a potential energy curve, characteristics of a chemical reaction of the molecule may be grasped. According to the information processing device 20 of the present embodiment as described above, since the deterioration in the accuracy of the VQE may be suppressed, a highly accurate potential energy curve may be obtained, which may contribute to drug discovery, new material development, and the like.

Note that, as described above, the processing contents described above may be implemented by causing the information processing device 20 to execute a program (for example, the quantum circuit weight reduction program).

The program may be recorded in a computer-readable recording medium (for example, the recording medium 26a). As the recording medium, for example, a magnetic disk, an optical disk, a magneto-optical disk, a semiconductor memory, or the like may be used. Examples of the magnetic disk include an FD and an HDD. Examples of the optical disk include a CD, a CD-recordable (R)/rewritable (RW), a DVD, and a DVD-R/RW. The program may be recorded in a portable recording medium and distributed. In that case, the program may be copied from the portable recording medium to another recording medium (for example, the HDD 23) and executed.

While one aspect of the quantum circuit weight reduction program, the information processing device, and the quantum circuit weight reduction method of the embodiments has been described above based on the embodiments, those are merely examples, and are not limited to the description above.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A non-transitory computer-readable recording medium storing a quantum circuit weight reduction program for causing a computer to execute processing comprising:
processing of performing, in ascending order of an interatomic distance, processing of calculating energy of a molecule by a variational quantum eigensolver (VQE) and acquiring a number of iterations of the VQE for a molecular structure of each of a plurality of interatomic distances among a plurality of atoms included in the molecule by using quantum circuit information that represents a second quantum circuit obtained by reducing a number of Rz gates for each angle parameter included in a first quantum circuit for weight reduction,
in which, in a case where a cumulative increase number of the number of iterations from a first number of iterations of a first interatomic distance among the plurality of interatomic distances to a second number of iterations of a second interatomic distance longer than the first interatomic distance among the plurality of interatomic distances becomes a threshold or more, the quantum circuit information to be used is changed to quantum circuit information that represents a third quantum circuit obtained by increasing the number of Rz gates for each angle parameter of the second quantum circuit, and the energy is calculated by the VQE for a molecular structure of a third interatomic distance longer than the second interatomic distance among the plurality of interatomic distances.

2. The non-transitory computer-readable recording medium according to claim 1, wherein the first quantum circuit includes a plurality of CNOT gates that accompanies the Rz gates, and when the quantum circuit information that represents the second quantum circuit is generated, the plurality of CNOT gates that accompanies the Rz gates deleted from the first quantum circuit is also deleted from the first quantum circuit.

3. The non-transitory computer-readable recording medium according to claim 1, wherein an initial value of the number of Rz gates for each angle parameter in the second quantum circuit is 1.

4. An information processing device comprising:
a memory configured to store quantum circuit information that represents a second quantum circuit obtained by reducing a number of Rz gates for each angle parameter included in a first quantum circuit for weight reduction; and
a processor coupled to the memory and configured to:
process of performing, in ascending order of an interatomic distance, processing of calculating energy of a molecule by a variational quantum eigensolver (VQE) and acquiring a number of iterations of the VQE for a molecular structure of each of a plurality of interatomic distances among a plurality of atoms included in the molecule by using the quantum circuit information;
in a case where a cumulative increase number of the number of iterations from a first number of iterations of a first interatomic distance among the plurality of interatomic distances to a second number of iterations of a second interatomic distance longer than the first interatomic distance among the plurality of interatomic distances becomes a threshold or more, change the quantum circuit information to be used to quantum circuit information that represents a third quantum circuit obtained by increasing the number of Rz gates for each angle parameter of the second quantum circuit; and
calculating the energy by the VQE for a molecular structure of a third interatomic distance longer than the second interatomic distance among the plurality of interatomic distances.

5. A quantum circuit weight reduction method for causing a computer to execute processing comprising:
processing of performing, in ascending order of an interatomic distance, processing of calculating energy of a molecule by a variational quantum eigensolver (VQE) and acquiring a number of iterations of the VQE for a molecular structure of each of a plurality of interatomic distances among a plurality of atoms included in the molecule by using quantum circuit information that represents a second quantum circuit obtained by reducing a number of Rz gates for each angle parameter included in a first quantum circuit for weight reduction,
in which, in a case where a cumulative increase number of the number of iterations from a first number of iterations of a first interatomic distance among the plurality of interatomic distances to a second number of iterations of a second interatomic distance longer than the first interatomic distance among the plurality of interatomic distances becomes a threshold or more, the quantum circuit information to be used is changed to quantum circuit information that represents a third quantum circuit obtained by increasing the number of Rz gates for each angle parameter of the second quantum circuit, and the energy is calculated by the VQE for a molecular structure of a third interatomic distance longer than the second interatomic distance among the plurality of interatomic distances.
